(19) United States
(12) Patent Application Publication
(10) Pub. No.: US 2005/0261605 A1
(43) Pub. Date: Nov. 24, 2005

(54) System for monitoring the health of an individual and method for use thereof

(75) Inventors: Yossef Shemer, Kiryat Tivon (IL); Zvi Herman, Haifa (IL)

Correspondence Address: MORGAN & FINNEGAN, L.L.P., 3 WORLD FINANCIAL CENTER, NEW YORK, NY, 10281-2101, US

(73) Assignee: S.A.E. Afikim Computerized Dairy Management System, Afikim (IL)

(21) Appl. No.: 10/531,698

(22) PCT Filed: Oct. 15, 2003
(86) PCT No.: PCT/IL03/00837

§ 371 Date: Jul. 26, 2005

Related U.S. Application Data
(60) Provisional application No. 60/419,282, filed on Oct. 18, 2002.

(51) Int. Cl. A61B 5/00; B65D 81/00

(52) U.S. Cl. 600/573; 600/584

(57) ABSTRACT

A medical analysis system for measuring properties and constituents of urine. The system includes a toilet unit mountable on a toilet bowl, readily attachable and detachable therefrom. The toilet unit includes a collection and sampling sub-unit for collecting and measuring the characteristics of urine and an electronics sub-unit for data collection and processing of the measured characteristics. The toilet unit also includes a communications link for data transfer between the collection and sampling and electronics sub-units. It also includes a means for removably fastening the collection and sampling sub-unit inside a toilet bowl, permitting ready attachment and removal of the toilet unit from a toilet bowl as desired. A wall unit, having a communications link for data transfer between the wall and toilet units, also includes means for storing and further processing the data for determining if changes have occurred in the measured characteristics of urine over time.

### FIELD OF THE INVENTION

The present invention relates to a system and method for monitoring the health of an individual by regular continual urine analyses.

### BACKGROUND OF THE INVENTION

Urinalysis is a long used and well known procedure for diagnosis of disease. Typically, modem day urinalysis is done in a laboratory setting using one or more instruments to determine the concentration of various urine constituents and the values of various urine properties. Such constituents and properties will often be referred to herein below as “characteristics of urine”. Often urinalysis is done using several different analytical techniques including, but not limited to, liquid chromatography, colorimetric analysis, polarography and voltammetry. Generally, laboratory analyzers are not portable and they are usable only at one site.

Today, for qualitative analysis and general monitoring of an individual's health in a non-laboratory setting, an individual uses chemically-treated dipsticks or diagnostic paper strips to determine if a constituent in the urine is present in abnormal concentrations. These dipsticks and diagnostic strips are typically single constituent analyzers. Generally, they require that the user physically collect and handle the excreted urine, followed by dipping the stick or strip into the urine.

For many individuals, particularly the aged and infirm, regular continual health monitoring is advantageous and often essential. Such monitoring will often require non-invasive daily, or even more frequent, monitoring of a plurality of constituents or other properties of an individual's urine. Recently, many systems have been proposed that have instruments placed inside or alongside a toilet or other types of sanitary installations which directly receive and measure parameters and constituents of urine as an individual excretes it from his body. These systems are almost always built into the sanitary installations, making them more expensive than conventional, commercially available systems.

Such prior art urinalysis systems can be found discussed in U.S. Pat. No. 5,184,359 to Tsukamura, et al; U.S. Pat. No. 5,111,539 to Hiruta, et al; U.S. Pat. Nos. 5,198,192 and 5,073,500 both to Saito, et al; U.S. Pat. Nos. 4,961,431 and 4,962,550 both to Ikenaga, et al; U.S. Pat. Nos. 5,720,054, 5,625,911, and 5,730,149 all to Nakayama, et al; and U.S. Pat. No. 4,636,474 to Ogura, et al . All these patents describe complicated systems that are built into, or are positioned in close proximity to, a toilet bowl or other sanitary installation. They are decidedly non-portable and intrinsically expensive.

U.S. Pat. No. 5,882,931 to Petersen discusses having measurement sensors in association with a member, the member fixedly glueable to a toilet, urinal, bidet or other sanitary installation.

A transportable, easily installable system for regular continual analysis of an individual's urine without requiring use of a laboratory would be desirable to monitor the health of individuals. It would be desirable to have a transportable system that could be used for analyzing and monitoring a plurality of urine properties and constituents—characteristics of urine—usable with many different individuals and many different sanitary installations. It would also be desirable to reduce the cost of such a system.

### SUMMARY OF THE PRESENT INVENTION

It is an object of the present invention to provide an indication of the state of health of an individual by monitoring changes in one or more characteristic constituents or properties of urine.

It is a further object of the invention to provide a system which is transportable, easily installable on, and detachable from, a plurality of toilets or other types of sanitary installations.

It is an object of the present invention to provide a system for measuring constituents and properties of urine usable with standard conventional sanitary installations.

It is another object of the invention to provide a system and method for measuring, analyzing and storing measurements of characteristic constituents and properties of urine samples obtained from different individuals.

It is another object of the invention to provide an individual with a method and system to monitor his health without having to handle his bodily wastes.

It is yet another object of the invention to provide a system which is relatively inexpensive.

The present invention provides a system which is easily installable in a toilet, urinal, bidet or other such sanitary installation. The system collects urine samples and analyzes constituents and properties of the excretion by use of light transmission and/or absorbance and/or reflectance and by measurement of electrical conductivity. In addition, the temperature and/or pH of the urine may also be measured.

Analyzed concentrations of urine constituents such as glucose, ketones, occult blood, electrolytes, and total dissolved solids (TDS), whose changes provide indications of a person's health or body condition, are collected and recorded. It is readily understood that the aforementioned constituents are exemplary only and they are not intended to be limiting.

A personal database of the urine properties and constituents measured is established for each individual using the toilet or other sanitary installation. Data is analyzed on the basis of the individual's last readings, generally a series of readings. An exception is indicated when there is a substantial deviation of the most recent measurement of an analyzed constituent or property from its average value, typically the average obtained over a predefined period. The average may be a moving average. While generally an average is used, other statistical measures, such as the standard deviation, may be used instead. Therefore when average is discussed herein it is to be considered as exemplary only and not to be considered limiting.

The system of the present invention is comprised of a “toilet unit” which includes inter alia a urine sampler, light sources and light detectors and a local main processing unit (MPU). Without being limiting, the MPU may be a microprocessor or microcontroller. A separate unit, generally, but not necessarily, mounted on a wall, herein designated as a “wall unit”, includes inter alia a user interface and a local MPU which stores and handles data transferred to a database. The MPU in the wall unit also may be a microprocessor or microcontroller. The toilet and wall units exchange information by a communications link.

For some populations of users, such as the institutionalized elderly or infirm, passive identification tags will be attached to the users to provide positive identification before use of the system. Attachment of such tags may be effected by any of several means, for example by means of a hand strap. In such cases, the wall unit will include a transceiver which will detect the tag and determine the identity of the user wearing it.

According to one aspect of the present invention, there is provided a medical analysis system for measuring one or more characteristics of urine of an individual. The system includes a toilet unit mountable on a toilet bowl so as to be readily attachable and detachable therefrom. The toilet unit includes a collection and sampling sub-unit for collecting and measuring the one or more characteristics of urine; an electronics sub-unit for data collection and processing of the measured one or more characteristics; a first communications link for facilitating data transfer between the collection and sampling sub-unit and the electronics sub-unit; and means for removably fastening the collection and sampling sub-unit inside a toilet bowl, operative to permit ready positioning of the sub-unit in and removal from any of a plurality of toilet bowls as desired. The system also includes a wall unit having a second communications link for facilitating data transfer between the wall and toilet units. The wall unit further includes means for storing and further processing the data to determine if changes have occurred in the one or more measured characteristics of urine.

In another embodiment of the system of the present invention, the system further includes output means operative in response to a change in the measured one or more characteristics of urine, the change being greater than a predetermined statistical threshold value, thereby to provide an output indication indicating that the individual requires further medical examination. The output means is chosen from a group consisting of a display displaying a text message; a display displaying a numerical value; an audio alarm; an alarm lamp; and a central institutional computer providing a text message.

In yet another embodiment of the system of the present invention, the means for removably fastening is a flexible strap, the strap in attachment with the collection and sampling and electronics sub-units, and positionable over a lip of the toilet bowl. In other embodiments the means for removably fastening is a rigid element, shaped to be positionable on a lip of a toilet, the rigid element in attachment with the collection and sampling sub-unit and the electronics sub-unit. In yet other embodiments the means for removably fastening is a hanger-like element, the element in attachment with the collection and sampling sub-unit and the electronics sub-unit, and shaped to be positionable on a lip of a toilet.

In another embodiment of the system of the present invention, the collection and sampling sub-unit includes one or more of the following elements: a sample cell; one or more light sources; one or more transmission detectors for detecting transmitted radiation from the light source; one or more absorption detectors for detecting absorbed radiation from the light source; one or more reflectance detectors for detecting reflected radiation from the light source; means for measuring conductivity; means for measuring temperature; and means for measuring pH.

Some embodiments of the system include a single light source and a plurality of detectors. In one of these embodiments the system includes a light source and a first and second detector, the first detector measuring transmission and the second detector measuring reflectance, the first detector generally positioned at an angle approximately 180° away from the light source and the second detector being positioned at any angle between about 0° and 90° from the light source.

In other embodiments of the system, the system includes a plurality of light sources and a plurality of detectors. In some of these embodiments, the system includes a first light source and a second light source, and a first and second detector, the first detector generally positioned at an angle approximately 180° away from the first light source and measuring transmission from the first light source and the second detector measuring reflectance from second light source, the second detector being positioned at any angle between about 0° and 90° from the second light source.

In yet other embodiments of the system, the system includes a plurality of light sources. In some of these embodiments the system includes a detector, a first light source generally positioned at an angle approximately 180° away from the detector, and a second light source positioned between about 0° and 90° from the detector, the detector functioning as a transmission detector with respect to the first light source and as a reflectance detector with respect to the second light source.

In still other embodiments the system includes a plurality of detectors.

In yet another embodiment of the system of the present invention, the collection and sampling sub-unit, the electronics sub-unit, and the communications link therebetween of the toilet unit are integrated into a single unit, the integrated unit removably fastened inside a toilet bowl.

In another embodiment of the system of the present invention, the electronics sub-unit includes one or more of the following elements: a main processing unit; a communications link to the wall unit; and an internal power source. The power source may be one or more batteries.

In yet another embodiment of the system, the wall unit includes one or more of the following elements: a main processing unit; a database memory block; a front panel display; a means for inputting information; an RFID transceiver; a power supply; a communications link to the toilet unit; and a communications link to a central institutional computer. The power supply here may be a battery power supply. In this embodiment the communications link between the wall unit and the central institutional computer includes a wired or wireless communications link. Also, in this embodiment the communications link between the wall unit and the toilet unit may be a wired or wireless communications link.

In yet another embodiment of the system of the present invention the wall unit is detachable, transportable and usable with a plurality of sanitary installations.

In another aspect of the present invention there is provided a method for measuring one or more characteristics of urine of an individual for determining if there is a deterioration in the health of the individual. The method includes the steps of: positioning a means for collecting and measuring one or more characteristics of urine in a toilet bowl; collecting urine passed by an individual within the means for collecting and measuring; measuring the value of the one or more characteristics of the collected urine; comparing the measured value of the one or more characteristics of the collected urine with a pre-determined statistical threshold for that one or more characteristics, the statistical threshold being calculated from a pre-determined series of similar measurements of that characteristic; and indicating that the measured value exceeds the statistical threshold of the one or more measured characteristics of urine, thereby to indicate changes in an individual's urine which may signal deterioration in the individual's health.

In an embodiment of the method of the present invention the method further includes a step of testing the reasonableness of the measured characteristic of the collected urine.

In yet another embodiment of the method of the present invention the method includes the step of identifying the user whose urine is to be collected and for which a measurement of one or more characteristics of urine is to be made.

In another embodiment of the method the step of measuring includes the step of measuring the concentration of one or more constituents of urine or the amount of one or more constituents of urine. In other embodiments the step of measuring includes the step of measuring the temperature, conductivity and/or pH of the urine.

In other embodiments of the present invention the step of comparing includes comparing the measured value of a characteristic of urine to a threshold statistical value based on an average of several similar measurements over a predetermined period of time or to a threshold statistical value based on the standard deviation of several similar measurements over a predetermined period of time or to a threshold statistical value based on a percentage of the average of several similar measurements over a predetermined period of time. In these embodiments the predetermined period of time may have starting and ending dates which may change, or the predetermined period of time is a fixed period having fixed predetermined starting and ending dates.

In yet another embodiment of the method of the present invention the step of comparing the measured value of the one or more characteristics of urine is a step of comparing at least two characteristics of urine, each of the characteristics having a different pre-determined statistical threshold.

### DEFINITIONS

Toilet—when used herein refers to any sanitary installation such as toilet, urinal, bidet or the like.
Toilet unit—when used herein refers to a unit that is constructed to be at least partially positionable in a toilet as defined above with suitable structural modifications for the type of sanitary installation being used.
Light source—when used herein is understood to include an electromagnetic radiation source which provides radiation in one or more of the following ranges: ultraviolet (UV), visible, and/or infrared (IR) ranges, both near (NIR) and far IR ranges. Moreover, whenever we are discussing light sources it is understood that it includes sources which can provide single wavelength (monochromatic) radiation and/or sources which provide a broad spectrum of wavelengths. Light emitting diodes (LEDs) are one type of light source which can be used. A single LED may be used if only one wavelength is required, or a cluster of LEDs, each covering a different part of the electromagnetic spectrum as discussed above, may be used.
Detector—when used herein may refer to a single detector for detecting a single wavelength or a cluster of detectors each detecting different portions of the electromagnetic spectrum.
Characteristic of urine—when used herein refers to a property of urine such as temperature, conductivity or pH, or a constituent of urine such as glucose, ketones, and electrolytes. These examples are to be considered non-limiting.

### BRIEF DESCRIPTION OF THE DRAWINGS

The present invention will be understood and appreciated more fully from the following detailed description taken in conjunction with the drawings in which:

FIG. 1 is a schematic perspective view of a toilet unit constructed according to an embodiment of the present invention, the toilet unit being mounted on a toilet;

FIG. 2 is a schematic side view of a toilet unit constructed according to the embodiment shown in FIG. 1, the unit being mounted on a toilet;

FIG. 3 is a block diagram of a toilet unit constructed according to an embodiment of the present invention;

FIG. 4 is a schematic diagram of the optical measurement system of a urine analyzer, constructed and operative according to a preferred embodiment of the present invention;

FIG. 5 is an enlarged view of the illumination rosette in the measurement system shown in FIG. 4;

FIGS. 6A and 6B are schematic drawings of a sampling cell according to another preferred embodiment of the present invention, for performing optical measurements on a sample of urine. FIG. 6A is a side view of the sampling cell, while FIG. 6B is a cross-sectional view;

FIG. 7 is a schematic view of the wall unit constructed according to an embodiment of the present invention;

FIG. 8 is a block diagram of a wall unit constructed according to an embodiment of the present invention; and

FIGS. 9A-9B present a flowchart of the method for monitoring health according to the present invention.

Similar elements in the Figures are numbered with similar reference numerals.

### DETAILED DESCRIPTION OF PREFERRED EMBODIMENTS

The present invention provides a portable system and method for periodic collection of urine from one or more individuals, sampling the urine and analyzing data obtained therefrom. The system analyzes the data collected over a predefined time period, typically several weeks. The system is easily transportable and usable with standard conventional commercially available sanitary installations on which it is easily installable. The system calculates mean values and/or other statistical measures for a plurality of constituents or other properties of a urine sample over the predefined period. The system indicates whether the measured and analyzed constituents and properties indicate that a change has occurred which exceeds a predetermined threshold value in one or more statistical measures of the analyzed urine constituents or properties. Correlations between different constituents and/or properties of the urine are also checked. Any changes and correlations exceeding the threshold values generates an exception report and/or an alert that brings the exception to the attention of medical professionals. These professionals determine whether a full medical examination is required. The above can be carried out for a plurality of individuals, the system having a means for identifying the source of the sample. The results of the analyzed urine are not intended to be used as a diagnostic tool but as an indicator that more detailed follow-up is required.

The system of the present invention includes two separate units: A. a toilet unit and B. a wall unit.

A. Toilet Unit

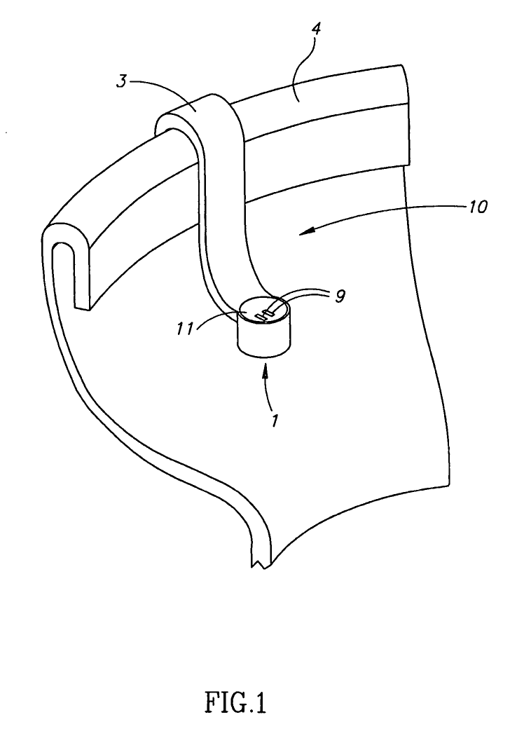

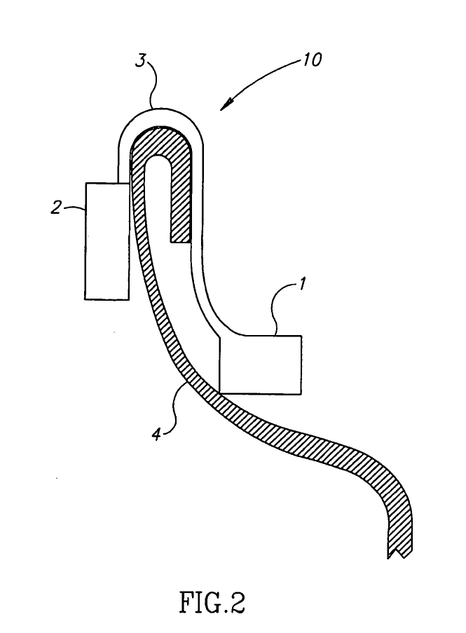

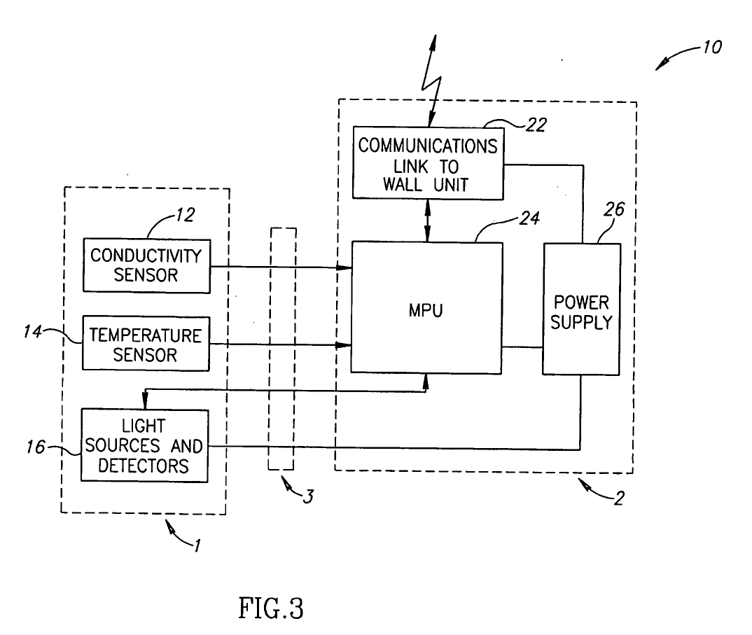

Reference is now made to FIGS. 1-3 which include a schematic perspective view of a portable toilet unit 10 constructed according to an embodiment of the present invention, toilet unit 10 being mounted on a toilet 4 (FIG. 1); a schematic side view of a toilet unit 10 constructed according to the embodiment shown in FIG. 1 mounted over the lip of a toilet 4 (FIG. 2); and a block diagram of a toilet unit 10 constructed according to an embodiment of the present invention (FIG. 3).

As best seen in FIGS. 1 and 2, portable toilet unit 10 is constructed to be non-permanently attachable, that is removably attachable, to the inside of a toilet 4. Toilet unit 10 includes a urine collection and sampling sub-unit 1 that includes a sampling cell (not shown), at least one light source and at least one light detector 16, conductivity sensors 12, typically conductivity electrodes, and a means for temperature measurement 14. These latter three elements are shown only in the block diagram of FIG. 3 and will be discussed further below. Urine enters the sampling cell through sample entrance holes 9, best seen in FIG. 1.

As seen in FIGS. 2 and 3, toilet unit 10 also includes an electronic sub-unit 2 which includes electronic circuits for data collection and processing, a first main processing unit (MPU) 24, and an internal power supply 26. Power supply 26 is typically, but not necessarily, a battery source. These latter elements are best seen in FIG. 3.

Between the toilet unit's 10 urine collection and sampling sub-unit 1 and its electronic sub-unit 2 is a communications link 3, typically at least a pair of wires embedded or otherwise attached to a flexible strap which permits the flow of information from sub-unit 1 to sub-unit 2. It also permits power to be supplied to sub-unit 1 from power supply 26 in sub-unit 2.

The flexible strap is constructed so that portability and non-permanent positioning of toilet unit 10 on a toilet 4 is possible. Collection and sampling sub-unit 1 of toilet unit 10 is positioned inside the toilet bowl while electronics sub-unit 2 is typically positioned outside the bowl 4. The positioning of collection and sampling sub-unit 1 is such that urine from the user directly impinges thereon. Sampling cell (not shown) of collection and sampling sub-unit 1 is purged of residual urine when a new urine sample enters the cell. It is also partially purged after each use by regular flushing of toilet 4. Because purging is not complete until a sufficient amount of a new urine sample is obtained, measurements are not made immediately after the detection of a new urine sample. Measurements are only begun after a sufficient quantity of the new urine sample has completely purged the sample cell of the old urine sample.

Communications link 3, in other embodiments, may be constructed as a wireless link. The flexible strap then would function solely as means to support and position the two sub-units 1 and 2 of toilet unit 10 in and/or on toilet 4.

The flexible strap represents only one means for removably fastening collection and sampling unit 1 inside a toilet. Other such means may include a rigid structure shaped to be positioned on a toilet so as to removably fasten the collection and sampling sub-unit 1 within a toilet bowl. Additionally, a hook or hanger-like element to which the collection and sampling sub-unit 1 is attached can also be used to position sub-unit 1 within a toilet bowl.

Collection and sampling sub-unit 1 includes a sampling cell (not shown), which allows optical measurements under dark conditions. The structure of collection and sampling sub-unit 1 is such as to allow urine to flow from a collection basin 11 through sampling cell entrance holes 9 and from there through a light trap (not shown) to a sampling cell where optical measurements are made. The urine then flows through another light trap (not shown) and out of the bottom (not shown) of collection and sampling sub-unit 1 via a narrow opening (not shown) there positioned.

The sampling cell (not shown) is constructed to include at least one light source and at least one light detector 16, herein after often denoted simply as “detector”, these latter shown in the block diagram of FIG. 3. The detectors can be selected and positioned so that light transmitted through (or absorbed by) the urine and/or light reflected from it can be collected and measured. In addition, the sampling cell may contain conductivity sensors 12, such as conductivity electrodes, that measure the electrical conductivity of the urine. A temperature sensor 14 also may be included in the sampling cell for measuring the temperature of the urine, thus providing an indication of the basal body temperature. The temperature will generally be compared to a set of prior temperature measurements. Comparison with a plurality of prior similar measurements ensures that high resolution in the temperature measurement is not required. The results of all the measurements are transmitted from collection and sampling unit 1 to electronics sub-unit 2 via communications link 3, the latter discussed above.

MPU 24 included in electronics sub-unit 2 of toilet unit 10, best seen in FIG. 3, is the heart of the electronic circuit of toilet unit 10. MPU 24 detects the presence of urine in the sampling cell. Once urine is detected, MPU 24 initiates analyses, typically electrical, temperature and optical analyses, of the sample. Optical analyses are used to calculate the concentration or amount of one or more urine constituents.

In other embodiments, sensors capable of measuring other urine properties, such as pH, may be included in collection and sampling sub-unit 1.

Electronics sub-unit 2 also includes a communications link 22, either wireless or wired, which is in communication with the wall unit discussed below. In addition, electronics sub-unit 2 includes a power supply 26 which provides power to MPU 24 and communications link 22 of electronics sub-unit 2 as well as to light sources and detectors 16 of collection and sampling sub-unit 1. Power supply 26 may be batteries or any other type of power supply.

The toilet unit of the present invention is intended to be used with standard conventional sanitary installations thereby providing cost savings. Using a means for removably fastening the collection and sampling unit inside a toilet allows the system to be easily transportable from one conventional sanitary installation to another.

Positioning of the light sources and detectors for obtaining data on the concentrations of constituents of urine, as well as analysis of such data, can be effected in a manner similar to that discussed in the Applicant's published PCT application WO 03/040704 entitled “Spectroscopic Fluid Analyzer”. Application WO 03/040704 is herein incorporated by reference in its entirety. In order to better instantiate the light source and detector issues involved, embodiments taken from the above referenced PCT application, modified as necessary, will now be presented.

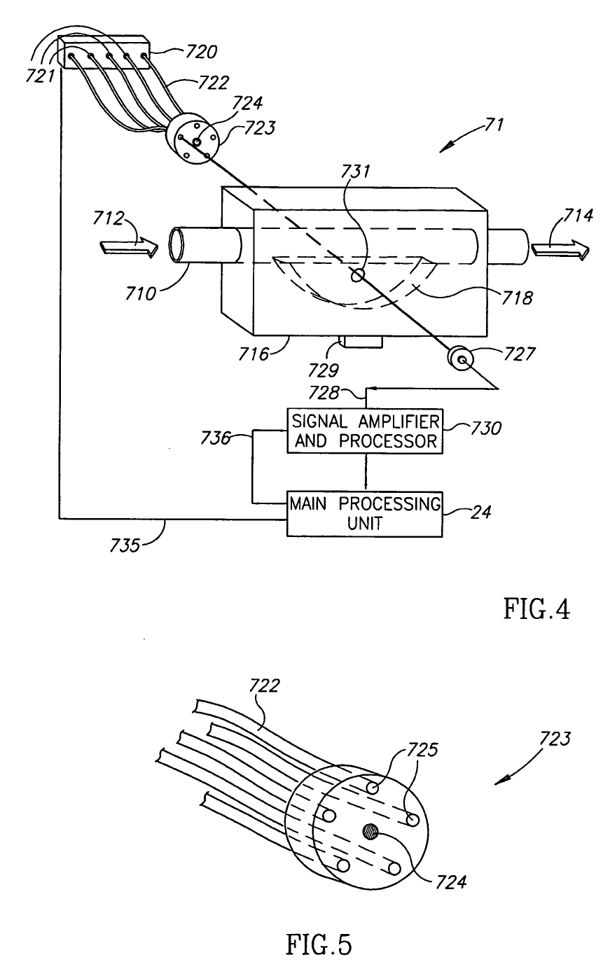

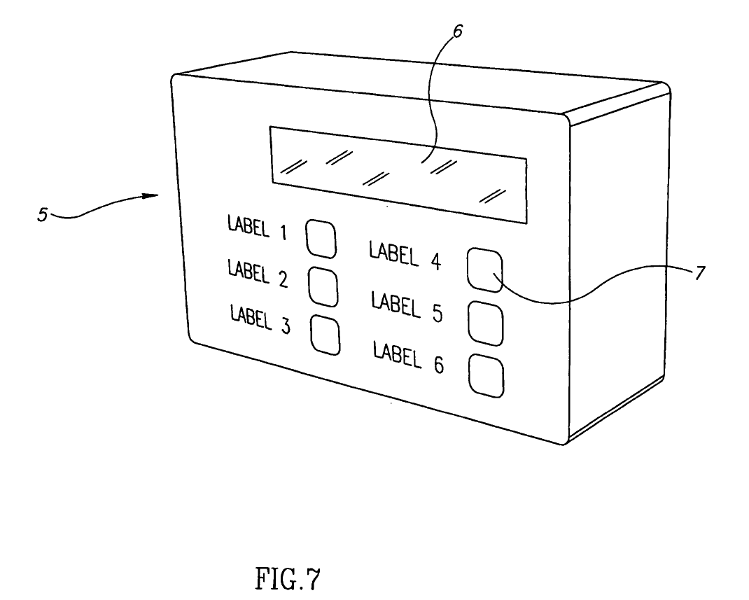

Reference is made to FIG. 4, which is a schematic illustration of a urine analyzer generally referenced 71 and its optical measurement system. Urine flows from an inlet 712 through a flow conduit 710 in a sampling cell towards an exit 714. A sampling cell 716 is located in urine flow conduit 710, and a sample of the flowing urine collects in the sample cavity 718. The structure and operation of cavity 718 are described in more detail hereinbelow, in connection with FIGS. 6A and 6B.

Adjacent to cavity 718 is a light emitting diode (LED) array 720 which preferably incorporates a number of discrete LED emitters 721, each emitting at a different wavelength within the range to be used for the measurement. According to one embodiment, the wavelengths of the LEDs 721 range from 260 nm to 950 nm covering the UV to NIR regions of the spectrum. According to this embodiment, the light output from each LED 721 is transmitted by means of an optical fiber 722 to a rosette 723, where all fibers 722 are bundled together to form a compact source, which emits the wavelength of whichever LED, or LEDs 721 desired. In FIG. 4, the LEDs are housed in array 720 without actually being shown and they are optically insulated from each other. Each LED illuminates directly only into its optical fiber. In the center of rosette 723 there is located a back-scattering detector 724, herein also referred to as a “reflectance detector”. Similarly, in the discussion herein below, when the terms “back-scattering”, “back-scattered” and the like are used, they also refer to the terms “reflecting”, reflected”, “reflectance” and the like. Power for the LEDs is provided by power supply 26 of electronics sub-unit 2 shown in FIG. 3.

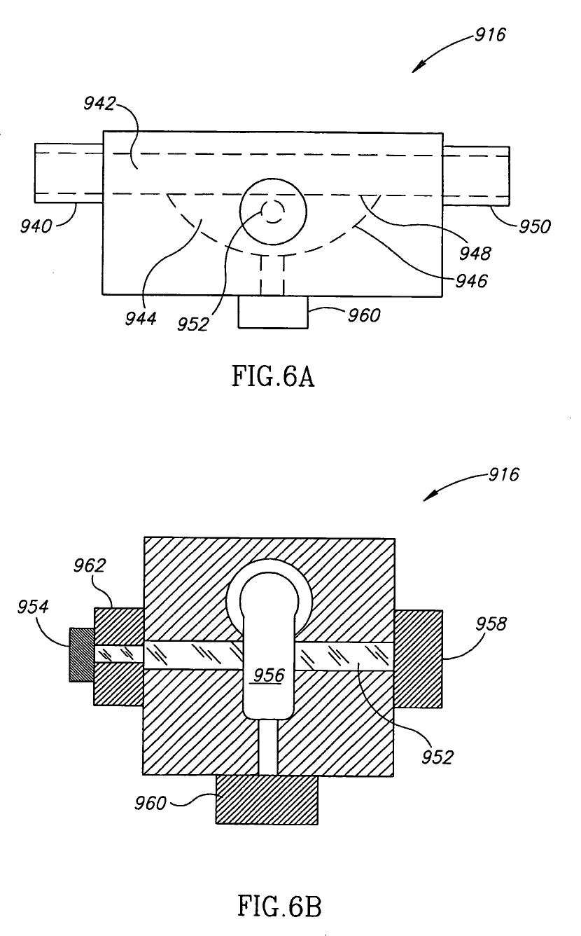

Reference is now made FIG. 5, where a schematic view of the illumination rosette 723 of FIG. 4 is presented, the rosette 723 being enlarged to show the details more clearly. Ends 725 of fibers 722, which emit the LED illumination, are grouped as closely as possible to each other, so that the different wavelength sources are as close as possible to a single source. At the center of this bundle is positioned detector 724, for use in detecting back-scattered (reflected) light. In operation, each of the LED's 721 generally is turned on sequentially, such that the spectrum of discrete wavelength points is measured sequentially. According to another preferred embodiment, another detector 729 (as seen in FIG. 4) may be positioned at an angle generally between 0° to 90°, here 90°, to the direction of the incident optical beam, so that scattering (reflecting) in directions other than that of back-scattered (reflection) can be detected.

While the embodiments of the optical system described herein above and below describe the optical system as using optical fibers, in other embodiments, systems employing light guides can also be constructed and used. This permits positioning back-scattering (reflectance) detector 724 away from rosette 723 and at various angles from the emitted light beams produced by LEDs 721, generally at angles of between 0 and 90 degrees from the emitted beam.

Referring again to FIG. 4, the light transmitted from source rosette 723 enters through an entry port (not shown) and passes through sample cavity 718 to be emitted through an exit port 731 to be detected, preferably by means of a silicon photo-detector 727 disposed adjacent to exit port 731. A detected signal 728 corresponding to the beam intensity is input into a signal amplifier (not shown in other Figures) and processor 730. This can optionally be operated as a phase sensitive detection system in order to provide optimum detection sensitivity, with the LED's modulated accordingly. The output intensities from the detection system are preferably fed to main processing unit 24 of FIG. 3, where the spectra obtained are analyzed. Main processing unit 24 passes control information 735 to LED sources 721 via communications link 3 of FIG. 3, to provide the modulation frequency, if used, and which is also input by means of control line 736 to the phase sensitive detector in signal amplifier and processor 730. Main processing unit (MPU) 24 also controls the switching order and timing of the LED sources 721, for scanning the complete spectral range to be measured.

According to one scanning program, each LED 721 is turned on for several milliseconds, and the absorption and/or scattering measurements are performed at that wavelength. In order to perform the measurements more rapidly, the transmission/absorbance signal on detector 727 and the back-scattered (reflected) signal on detector 724 are measured simultaneously. If a right-angled detector 729 is used, its signal is also measured simultaneously with the signals on detectors 724 and 727. 5 In another embodiment, the fiber optic cables of the light source and detector elements of the system may be replaced with plastic light guides, forming a less costly, more compact, and waterproof assembly.

In order to overcome physical size constraints, the 10 LED's 721 are preferably divided into two groups of five, each rosette 723 having only 5 LED's 721 and its own central detector 724, and each rosette 723 being disposed on opposite sides of sample cavity 718. According to this embodiment, illumination and detection is performed sequentially from both sides of sampling cavity 718, with the 5 LED's 721 in each rosette 723. Detector 724 of each rosette collects back-scattered light from its own LED's 721 and transmitted light from the LED's 721 of the opposite rosette 723. According to this embodiment, the function of detector 729, if used, remains unchanged.

A major problem in analyzing the spectrum of a multi-component fluid such as urine arises from the overlap of the individual absorption and scattering spectra of each of the separate components. Furthermore, according to the illumination method described herein above where the spectral width of the LED sources 721 may be such as to include a number of such individual absorption and scattering spectral lines, in order to quantitatively analyze the urine for its separate constituents, a method must be provided for extracting this information about the identification of the lines present, and determination of their intensity. The method must be capable of doing this to a plurality of lines “hidden” within the intensity measurements obtained from the relatively broad bandwidth LED sources 721.

According to one method, a high order polynomial expression is used to express the concentrations, C, of the various urine constituents in sample cavity 718 in terms of the measured transmitted and reflected (scattered) light intensities for each LED measurement, each intensity term appearing with an empirical coefficient. According to one embodiment, the polynomial may be of the form:
C %=ΣaxₜⱼIₜⱼ+ΣbxₜⱼI²ₜⱼ+ΣcxₜⱼI³ₜⱼ+ . . . +ΣaxᵣⱼIᵣⱼ+ΣbxᵣⱼI²ᵣⱼ+ΣcxᵣⱼI³ᵣⱼ+ . . .
where:
j=1-10, representing 10 discrete light sources in the UV, NIR and visible spectrum;
Iₜⱼ=intensity of the light from source j, detected on the transmittance photo-detector;
Iᵣⱼ=intensity of the light from source j, detected on the reflectance photo-detector;
C %=concentration of constituent C; and
axₜⱼ, bxₜⱼ, cxₜⱼ . . . , axᵣⱼ, bxᵣⱼ, cxᵣⱼ . . . , =empirical coefficients, relating the intensities of the light detected to the concentration of the constituent C. According to one embodiment, a third order polynomial is used, and only coefficients up to cxₜⱼ and cxᵣⱼ are used.

The values of these empirical coefficients are initially experimentally determined preferably by using a statistical analysis method, such as by performing Partial Least Squares (PLS) regression or Ridge Least Square (RLS) calculations on a large database of absorption and reflectance data acquired experimentally from a large number of urine samples having different and variable constituents. To provide a sufficiently broad database, the samples are preferably obtained from several hundred different measurements. The data are obtained from absorption and reflectance measurements made using the light emitted from the ten LED's. The constituents of each sample of urine are independently determined, preferably using a standard spectrophotometric method, and these known constituent concentrations are then used to extract the empirical coefficients by using a preferred statistical analysis method.

Once these coefficients are known they are stored in main processing unit 24, along with the concentrations of the sample with which they are associated, as a reference database in the computing system memory for use in measurements of unknown samples. The extraction of the concentrations of the constituents from an unknown sample of urine is preferably performed by a further statistical analysis method, comparing the measured intensities with the contents of the database, such as is known from chemometric analysis methods used in the analysis of multiple component chemical reaction dynamics. According to one preferred embodiment of the present invention, the analyzer uses ten LED sources 721, such that 20 measurement signals are obtained from each unknown sample of urine, 10 from transmission measurements, one from each of the 10 LED's 721, and 10 from reflectance or back-scattering measurements, one from each of the 10 LED's 721. These 20 measurement signals, each at their known wavelength range, are then related, preferably by means of the statistical analysis chemometric-type methods, to a large database of stored spectral curves related to various urine compositions, and from the analysis a unique set of concentrations of the constituents of the urine sample is determined. This method of calibration and analysis thus allows the use of inexpensive LED's 721 with their non-uniform wide spectral range as light sources, rather than a more discrete and monochromatic source of light, such as a laser, as is used in some prior art optical fluid analyzers.

Reference is now made to FIGS. 6A and 6B, which show schematic drawings of a sampling cell 916 for performing optical measurements on a flowing urine sample, constructed and operative according to another embodiment. FIG. 6A is a side view of the sampling cell. Urine enters sampling cell 916, directly from collection basin 11 in FIG. 1, by means of sample entrance holes 9 (as seen in FIG. 1) via a light trap. Urine then flows along the main flow conduit 942, entering sampling cell 916 via inlet conduit 940. Adjoining the main flow conduit 942 is a recessed sample cavity 944, having a smooth profile 946 preferably in the shape of an arc of a circle. Cavity 944 is in fluid flow contact with main flow conduit 942 along a side wall 948 of the flow conduit 942. Urine flows out of sampling cell 916 by means of an exit conduit 950. During use, sampling cell 916 is orientated such that sampling cavity 944 is located in a generally downwards direction. As a result, cavity 944 fills with a sample of the flowing urine. Each new flow of urine entering cavity 944 purges the previous sample, replacing it with a new sample.

Disposed generally near the center of sampling cavity 944, and below wall 948 of the flow conduit 942, there is situated an orifice 952 in the body of the urine sampling cell 916, through which an optical beam enters sampling cavity 944. According to the preferred embodiment, orifice 952 is filled with an optically transparent solid through which the optical beam passes, thereby preventing flow of urine from orifice 952. Alternatively, light guides (not shown) disposed where orifice 952 meets sampling cavity 944 may be used for this purpose. On one side of orifice 952 there is a light source mount 954 in which, or to which, is attached a LED illumination rosette, similar to rosette 723 of FIGS. 4 and 5, the latter providing an incident optical beam. The optical beam traverses the central area 956 of recessed cavity 944 and orifice 952. Remote from the light source rosette and mounted to light source mount 954 is a detector mount 958 in which, or to which, is installed a transmission detector, similar to detector 727 (seen in FIG. 4).

According to a further preferred embodiment of the present invention, in addition to the transmission measurements, back-scattering (reflectance) measurements may be achieved by use of a detector located as close to the light source as possible. This is achievable by mounting a detector at the center of the rosette, similar to detector 724 in rosette 723 of FIGS. 4 and 5, and by attaching it to sampling cell 916 using mount assembly 962. According to yet another embodiment of the present invention, the optical measurement may be made by scattering (reflectance) other than back-scattering, and for this purpose another detector mount 960 is disposed, preferably at right angles to the optical beam path, to detect light scattered at 90 degrees by urine in sampling cavity 944. According to yet another preferred embodiment of the present invention, transmission detector 958 is replaced by another rosette similar to rosette 723 of FIGS. 4 and 5 including both multiple light sources and a detector, as described above.

It should be noted that the above instantiation of the light sources and detectors is only exemplary and not to be considered limiting.

B. Wall Unit

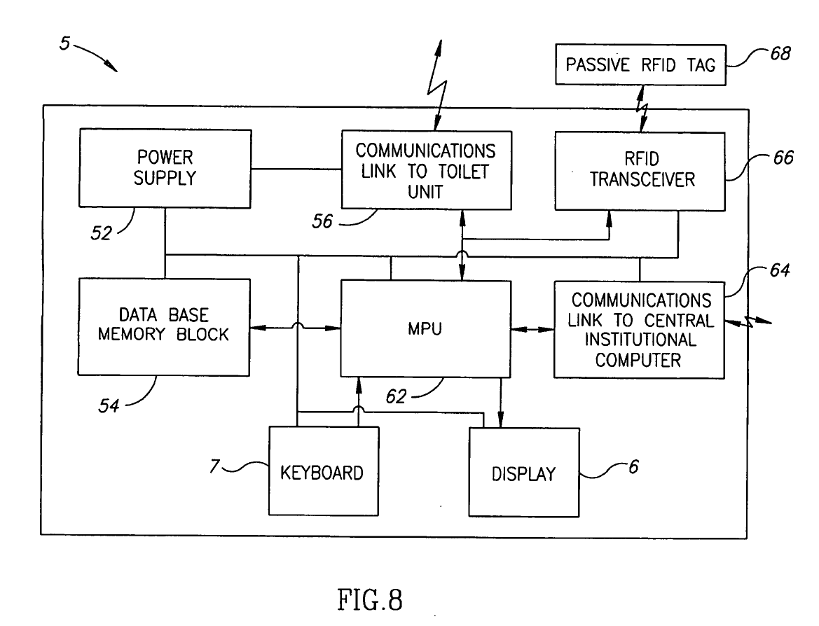

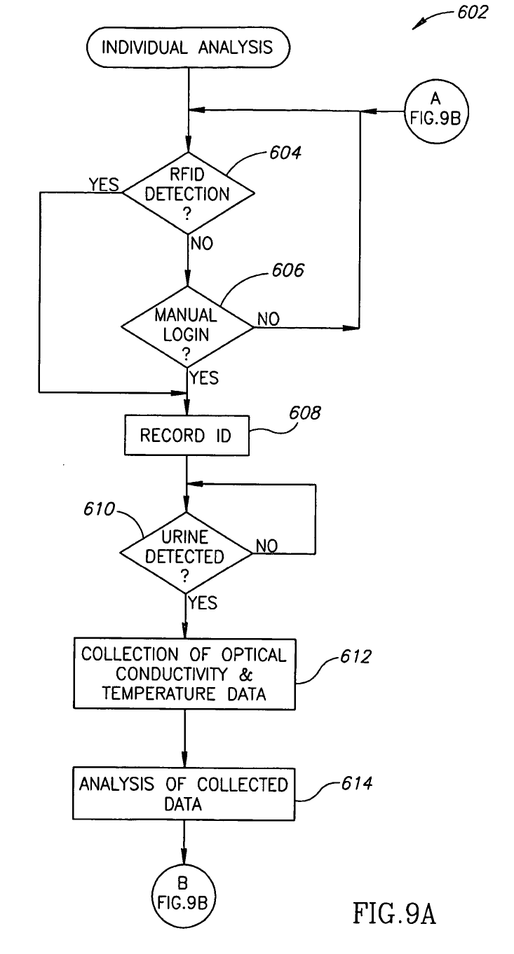

Wall unit 5, best seen in FIG. 7 and in the block diagram of FIG. 8 to which reference is now made, is a wall mountable panel, easily and simply installable, including a central second main processing unit (MPU) 62, a memory block for accommodating a database 54, a communications link 56 to toilet unit 10, a front panel with display 6 and a means for inputting information such as a keyboard 7 for interface with the user.

Communications link 56 transfers data from toilet unit 10 (as seen in FIG. 3) to MPU 62 of wall unit 5 and instructions from MPU 62 to toilet unit 10. Communications link 56 may be a wireless or wired link.

All elements of wall unit 5 are powered by a power supply 52 shown in FIG. 5, which may include either batteries or an internal mains power supply.

In some embodiments, a radio frequency identification (RFID) transceiver 66 may be included in wall unit 5 as shown in FIG. 8. Transceiver 66 may be used to read passive user identification tags 68 often provided to sick, elderly or otherwise partially or totally incapacitated individuals. When installed in institutions, a second communications link 64 may be provided for communicating between wall unit 5 and the central institutional computer (not shown).

While we have described wall units as being a wall mountable panel, in some embodiments it may be positioned anywhere within communication distance of toilet unit 10. Moreover, while the wall unit can be mountable on a wall, it also can be detachable therefrom, permitting it to have transportable character.

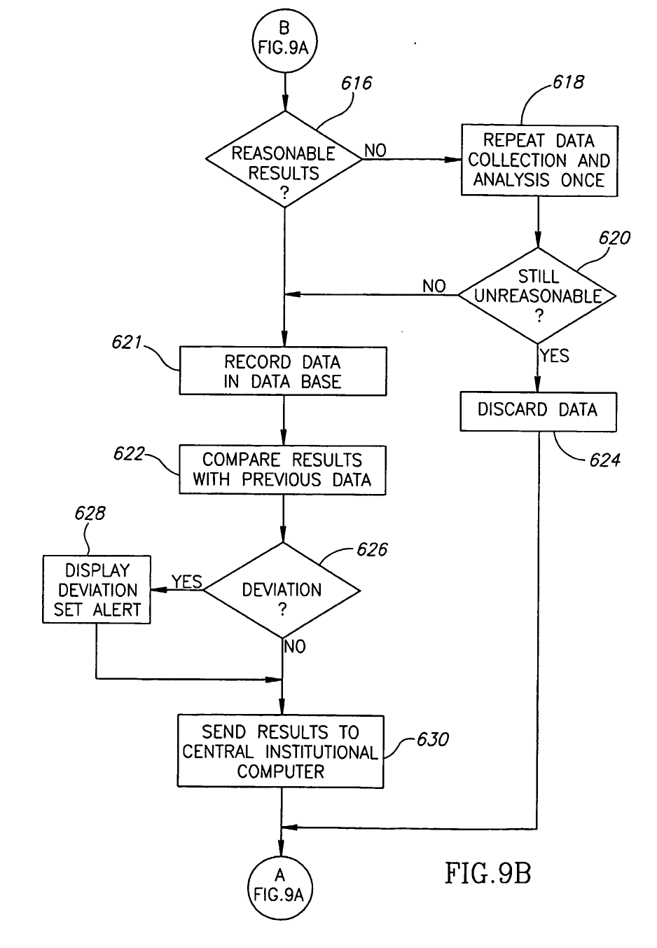

The results of the analyses made in MPU 24 of electronics sub-unit 2 of toilet unit 10 (as seen in FIG. 3) are communicated to MPU 62 in wall unit 5 via communications link 56. MPU 62 of wall unit 5 keeps track of the date and time of all the urine analyses. Data collected by and received from toilet unit 10 is transferred by MPU 62 to a database in database memory block 54. Data is also retrievable from memory block 54 by MPU 62. A database is opened for each individual user. Date and time data will be recorded together with an individual's analyzed urine data and stored in the database in memory block 54. The system processes data for each registered user according to the method described below in conjunction with FIGS. 9A-9B.

As described above, results of the measurements of urine constituent concentrations and urine properties, such as conductivity and temperature, are conveyed from toilet unit 10 to wall unit 5, the latter unit maintaining the database for all individuals using toilet unit 10. Prior to entering data into the database, the data is analyzed to verify that it is reasonable. Extreme values are ignored by MPU 62 in wall unit 5, assuming that these values reflect artifacts of the measuring process.

Data is kept in database memory block 54 for a predetermined length of time. Typically, but without being limiting, this period can be 14 days. Data entered into the database relating to a period prior to the predetermined length of time will be discarded.

As noted previously, data is collected on an individual by individual basis regarding urine constituents, such as, but not limited to, sugar, ketones, occult blood, electrolytes, TDS (total dissolved solids), and other selected constituents whose changes provide indications of a person's health or body condition. Without being limiting, and solely for exemplary purposes, these other constituents may include hormonal or other indicators of pregnancy or ovulation.

Data will be collected whenever a registered person identifies himself by using an input device such as keyboard 7 in wall unit 5 (FIG. 7). Alternatively, when ID tags 68 are used, such as with aged or incapacitated individuals, identification of a registered person will be performed automatically by wall unit 5 typically by using radio frequency identification (RFID) transceiver 66, best seen in FIG. 8 as is well known to those skilled in the art.

Each time that a person logs in, the reference value of each urine constituent is recalculated using the average of all the values for a predetermined period prior to the actual date. Upper and lower thresholds are determined for each constituent; these are typically defined as a percent of the average value, which then may be translated into absolute values of the threshold. Threshold values based on other statistical measures such as standard deviation may also be used.

Measured values are compared to the upper and lower threshold values. If a measured value lies outside the threshold values, an indication may be provided to the user on display 6 of wall unit 5. The indication is registered as an exception in the database. Alternatively, or additionally, the exception may be conveyed to a member of a medical staff for immediate attention. Each registered individual may be able to browse the database, viewing the results of his samples over the last day and/or a predefined prior period, as well as any recorded exceptions stored therein.

When installed in institutions, such as hospitals, old age homes and the like, MPU 62 of wall unit 5 may be linked by communications link 64 to a central institutional computer (not shown). Communications link 64 may be a wireless or a wired link. The central institutional computer uses a software package which collects data, maintains a central database and can provide alerts when an extreme value for a urine constituent or urine property is received for an individual.

While the indication, also herein described as an exception report, has been described above as being transmitted to a user through display 6 of wall unit 5, other means of transmittal are also available. Exception reports (or alerts) may be transmitted either as a numerical or textual display on display of wall unit 5 (FIG. 7) an alarm lamp (not shown) on wall unit 5 or elsewhere, or an audio alarm (not shown) on wall unit 5 or elsewhere. Additionally, exceptions can be printed out at the central institutional computer (not shown) when wall unit 5 is in communication with such a system as shown in FIG. 8.

In another embodiment of the present invention, the collection and sampling sub-unit and the electronics sub-unit of the toilet unit, as well as the communications link between them, can be integrated into a single unit and non-permanently positioned within a toilet bowl. A hanger-like attachment can be used to hang, suspend or position the integrated toilet unit in the toilet bowl. The integrated unit will be positioned so that the urine from an individual directly impinges thereon. The electronics subunit and communications link of this integrated toilet unit are packaged so that the liquid does not impair function.

Reference is now made to FIGS. 9A-9B which include a flowchart showing the method 602 of the present invention.

An individual who is to use the system of the present invention and for whom an analysis of his urine is to be done, logs on to the system. This may be effected either electronically 604 (here using an RFID identification tag) or manually 606. If an RFID tag is detected 604 by the system, the individual is identified and the system readies itself to detect the presence of urine. If no ID tag is detected, the system attempts to determine if a log in has been effected manually 606. If a manual log in is detected, the individual is identified and the system readies itself to detect the presence of urine. It should be noted that not all systems have RFID tag identification or other electronic user identification capabilities, but all systems have manual identification capabilities.

Once an individual is identified manually 606 or electronically 604, the identification data is recorded 608 in a main processing unit and the system readies itself to detect the presence of urine 610. If no urine is detected, the system keeps searching until the system times out. If urine is detected, the system's light sources and detectors, conductivity, temperature and other sensors are activated and data is collected 612 from measurements made on the urine sample. The collected data is then analyzed 614 by a main processing unit.

The results of the measured and analyzed data are then checked 616 for reasonableness. If the results are reasonable the measured data is recorded 621 in a database, the data being associated with the previously identified individual. If the results are unreasonable, data collection and analysis is repeated 618. If the results are still unreasonable 620, the data is discarded 624 and the system exits waiting for another individual to be identified by manual 606 or electronic 604 identification, as described above. If the second set of analyzed data collected is reasonable 620 the data and its analysis is recorded 621 in a database, the date being associated with the identifier data of the individual. Not specifically shown is that the time and date of sampling is also associated with the stored, analyzed data by the main processing unit.

The recorded 621 data in the database is compared 622 with the data previously stored in the database. A determination is made as to whether the most recently acquired data deviates 626 from previously stored data. If a deviation is noted, an alert or exception report is generated and/or displayed 628. The alert or exception report is then stored in the data base associated with the examined individual and then transmitted 630 to the central institutional computer. If no deviation is found 626, the data is transmitted 630 directly to the central institutional computer without generating an alert or exception report.

It should be noted that the flowchart shows sending data to a central institutional computer. This is an optional step and possible only when the system of the present invention is applied in a large institution. When the system is not being used in an institution, the system returns to the beginning of the flowchart after storing the deviation in the data base associated with the examined individual.

In all cases, after the system has stored 630 the results in the database of the system, the system prepares for the identification, either manually 606 or electronically 604, of a new individual (user) and analyzes the individual's urine sample as described above and shown in FIGS. 9A-9B.

It will be appreciated by persons skilled in the art that the present invention is not limited by what has been particularly shown and described herein above. Rather the scope of the invention is defined by the claims that follow:

1. A medical analysis system for measuring at least one characteristic of urine of an individual, said system including:
a toilet unit mountable on a toilet bowl so as to be readily attachable and detachable therefrom, said portable toilet unit including:
a. a collection and sampling sub-unit for collecting and measuring the at least one characteristic of urine;
b. an electronics sub-unit for data collection and processing of the measured at least one characteristic;
c. a first communications link for facilitating data transfer between said collection and sampling sub-unit and said electronics sub-unit; and
d. means for removably fastening said collection and sampling sub-unit inside a toilet bowl, operative to permit ready positioning of said sub-unit in and removal from any of a plurality of toilet bowls as desired; and
a wall unit having a second communications link for facilitating data transfer between said wall unit and said toilet unit, said wall unit further including means for storing the data, and for further processing the data to determine if changes have occurred in the at least one measured characteristic of urine.

2. A system according to claim I wherein said system further includes output means operative in response to a change in the measured at least one characteristic of urine, the change being greater than a predetermined statistical threshold value, thereby to provide an output indication indicating that the individual requires further medical examination.

3. A system according to claim 2 wherein said output means is chosen from a group consisting of
a display displaying a text message;
a display displaying a numerical value;
an audio alarm;
an alarm lamp; and
a central institutional computer providing a text message.

4. A system according to claim 1, wherein said means for removably fastening is a flexible strap, said strap in attachment with said collection and sampling sub-unit and said electronics sub-unit, and positionable over a lip of the toilet bowl.

5. A system according to claim 1, wherein said means for removably fastening is a rigid element, shaped to be positionable on a lip of a toilet, said rigid element in attachment with said collection and sampling sub-unit and said electronics sub-unit.

6. A system according to claim 1, wherein said means for removably fastening is a hanger-like element, said element in attachment with said collection and sampling sub-unit and said electronics sub-unit, and shaped to be positionable on a lip of a toilet.

7. A system according to claim 1 wherein said collection and sampling sub-unit includes at least one of the following elements:
a sample cell;
at least one light source;
at least one transmission detector for detecting transmitted radiation from said light source;
at least one absorption detector for detecting absorbed radiation from said light source;
at least one reflectance detector for detecting reflected radiation from said light source;
means for measuring conductivity;
means for measuring temperature; and
means for measuring pH.

8. A system according to claim 7 wherein said system includes a single light source and a plurality of detectors.

9. A system according to claim 8 wherein said system includes a light source and a first and second detector, said first detector measuring transmission and said second detector measuring reflectance, said first detector generally positioned at an angle approximately 180° away from said light source and said second detector being positioned at any angle between about 0° and 90° from said light source.

10. A system according to claim 7 wherein said system includes a plurality of light sources and a plurality of detectors.

11. A system according to claim 10 wherein said system includes a first light source and a second light source, and a first and second detector, said first detector generally positioned at an angle approximately 180° away from said first light source and measuring transmission from said first light source, and said second detector measuring reflectance from said second light source, said second detector being positioned at any angle between about 0° and 90° from said second light source.

12. A system according to claim 7 where there is a plurality of light sources.

13. A system according to claim 12 wherein said system includes a detector, a first light source generally positioned at an angle approximately 180° away from said detector, and a second light source positioned between about 0° and 90° from said detector, said detector functioning as a transmission detector with respect to said first light source and as a reflectance detector with respect to said second light source.

14. A system according to claim 7 wherein said system includes a plurality of detectors.

15. A system according to claim 1 wherein said collection and sampling sub-unit, said electronics sub-unit, and said communications link therebetween of said toilet unit are integrated into a single unit, said integrated unit removably fastened inside the toilet bowl.

16. A system according to claim I wherein said electronics sub-unit includes at least one of the following elements:
a main processing unit;
a communications link to said wall unit; and
an internal power supply.

17. A system according to claim 16 wherein said power supply is a battery.

18. A system according to claim I wherein said wall unit includes at least one of the following elements:
a main processing unit;
a database memory block;
a front panel display;
a means for inputting information;
an RFID transceiver;
a power supply;
a communications link to said toilet unit; and
a communications link to a central institutional computer.

19. A system according to claim 18, wherein said communications link between said wall unit and said central institutional computer includes a wireless communications link.

20. A system according to claim 18, wherein said communications link between said wall unit and said central institutional computer includes a wired communications link.

21. A system according to claim 18, wherein said communications link between said wall unit and said toilet unit includes a wireless communications link.

22. A system according to claim 18, wherein said communications link between said wall unit and said toilet unit includes a wired communications link.

23. A system according to claim 18, wherein said power supply includes a battery power supply.

24. A system according to claim 1, wherein said wall unit is detachable, transportable and usable with a plurality of sanitary installations.

25. A method for measuring at least one characteristic of urine of an individual for determining if there is a deterioration in the health of the individual, wherein said method includes the steps of:
positioning a means for collecting and measuring at least one characteristic of urine in a toilet bowl;
collecting urine passed by an individual within the means for collecting and measuring;
measuring the value of the at least one characteristic of the collected urine;
comparing the measured value of the at least one characteristic of the collected urine with a pre-determined statistical threshold for that at least one characteristic, the statistical threshold being calculated from a pre-determined series of similar measurements of that characteristic; and
indicating that the measured value exceeds the statistical threshold of the at least one measured characteristic of urine, thereby to indicate changes in an individual's urine which may signal deterioration in the individual's health.

26. A method according to claim 25 further comprising a step of testing the reasonableness of the measured characteristic of the collected urine.

27. A method according to claim 25 further comprising the step of identifying the user whose urine is to be collected and for which a measurement of at least one characteristic of urine is to be made.

28. A method according to claim 25 wherein said step of measuring includes the step of measuring the concentration of at least one constituent of urine.

29. A method according to claim 25 wherein said step of measuring includes the step of measuring the amount of at least one constituent of urine.

30. A method according to claim 25 wherein said step of measuring includes the step of measuring the temperature of the urine.

31. A method according to claim 25 wherein said step of measuring includes the step of measuring the conductivity of the urine.

32. A method according to claim 25 wherein said step of measuring includes the step of measuring the pH of the urine.

33. A method according to claim 25 wherein said step of comparing includes comparing the measured value of a characteristic of urine to a threshold statistical value based on an average of several similar measurements over a predetermined period of time.

34. A method according to claim 25, wherein said step of comparing includes comparing the measured value of a characteristic of urine to a threshold statistical value based on the standard deviation of several similar measurements over a predetermined period of time.

35. A method according to claim 25 wherein said step of comparing includes comparing the measured value of a characteristic of urine to a threshold statistical value based on a percentage of the average of several similar measurements over a predetermined period of time.

36. A method according to any one of claims 33-35 wherein the predetermined period of time has starting and ending dates which may change.

37. A method according to any one of claims 33-35 wherein the predetermined period of time is a fixed period having fixed predetermined starting and ending dates.

38. A method according to claim 25 wherein said step of comparing the measured value of the at least one characteristic of urine is a step of comparing at least two characteristics of urine, each of the characteristics having a different pre-determined statistical threshold.
